(19) United States
(12) Patent Application Publication
(10) Pub. No.: US 2013/0216485 A1
(43) Pub. Date: Aug. 22, 2013

(54) Dentifrice Composition with Reduced Astringency

(71) Applicants: Thomas S. Campbell, Flemington, NJ (US); Steven W. Fisher, Middlesex, NJ (US); Michael Prencipe, Princeton Junction, NJ (US)

(72) Inventors: Thomas S. Campbell, Flemington, NJ (US); Steven W. Fisher, Middlesex, NJ (US); Michael Prencipe, Princeton Junction, NJ (US)

(73) Assignee: Colgate-Palmolive Company, Piscataway, NJ (US)

(21) Appl. No.: 13/882,494

(22) PCT Filed: Nov. 4, 2010
(86) PCT No.: PCT/US10/55389

§ 371 Date: Apr. 29, 2013

(51) Int. Cl. A61K 8/55; A61K 8/81; A61Q 11/00

(52) U.S. Cl. CPC A61K 8/55; A61Q 11/00; A61K 8/8111
USPC 424/52; 424/49; 424/57; 424/55

(57) ABSTRACT

A dentifrice composition containing, in combination, an orally acceptable vehicle; a halogenated diphenyl ether; a soluble zinc salt; and a chelating agent to reduce astringency.

### BACKGROUND

Zinc ions are known to be effective anti-microbial agents. These ions provide anti-gingivitis and anti-plaque benefits and may also improve breath and reduce sensitivity. In particular, zinc has been shown to have anti-plaque, anti-gingivitis and anti-tartar efficacy. In addition, zinc has also shown its efficacy as an anti-malodor agent.

Dentifrice compositions have been developed that provide multiple therapeutic benefits by combining zinc with other actives in a single composition. However, dentifrice compositions containing, in combination, a high level of soluble zinc and halogenated diphenyl ether as an antibacterial enhancing agent (e.g., triclosan) may have an unpleasant flavor to consumers due to an increase in astringency.

### SUMMARY

One aim of the present invention is to provide a dentifrice composition containing, in combination, a halogenated diphenyl ether; a soluble zinc salt; and a chelating agent to reduce astringency. Another aim of the present invention is to provide such a dentifrice composition containing triclosan.

A further aim of the present invention is to provide such a dentifrice composition containing a chelating agent selected from the group consisting of: gluconates, citrates, tartrates, anionic polymeric carboxylates, polyvinyl phosphonates, and phytates to reduce astringency.

A still further aim of the present invention is to provide a method for the treatment and prevention of bacterial plaque accumulation including administering to the oral cavity a dentifrice composition containing, in combination, a halogenated diphenyl ether; a soluble zinc salt; and a chelating agent.

### DETAILED DESCRIPTION

As used throughout, ranges are used as a shorthand for describing each and every value that is within the range. Any value within the range can be selected as the terminus of the range. In addition, all references cited herein are hereby incorporated by reference in their entireties. In the event of a conflict in a definition in the present disclosure and that of a cited reference, the present disclosure controls.

There is a desire to utilize zinc in combination with agents such as triclosan to provide improved efficacy over current antibacterial enhancing agent-containing dentifrice formulations in the areas of tartar control, fresh breath benefits and plaque/gingivitis reduction.

As will be demonstrated herein, the preferred embodiments of the present invention can provide a dentifrice that provides multiple therapeutic benefits by combining zinc ions, e.g. as zinc citrate, and triclosan in combination with an chelating agent to reduce astringency.

Unless otherwise specified, all percentages and amounts expressed herein and elsewhere in the specification should be understood to refer to percentages by weight, and all measurements are made at 25° C. The amounts given are based on the active weight of the material. The recitation of a specific value herein, whether referring to respective amounts of components, or other features of the embodiments, is intended to denote that value, plus or minus a degree of variability to account for errors in measurements. For example, an amount of 10% may include 9.5% or 10.5%, given the degree of error in measurement that will be appreciated and understood by those having ordinary skill in the art. All percentages used herein are by weight of the dentifrice composition, unless otherwise specified. All unless otherwise specified.

Herein, “effective amount” means an amount of a compound or composition sufficient to significantly induce a positive benefit, preferably an oral health benefit, but low enough to avoid serious side effects, i.e., to provide a reasonable benefit to risk ratio, within the sound judgment of a skilled artisan.

The dentifrice composition of the present invention may be in the form of a toothpaste or dentifrice. The term “dentifrice”, as used herein, means paste or gel formulations unless otherwise specified. The dentifrice composition may be in any desired form, such as deep striped, surface striped, multi-layered, having the gel surrounding the paste, or any combination thereof.

The dentifrice composition is a product, which in the ordinary course of administration, is not intentionally swallowed for purposes of systemic administration of particular therapeutic agents, but is rather retained in the oral cavity for a time sufficient to contact substantially all of the tooth surfaces and/or oral tissues for purposes of oral activity.

The term “carrier” as used herein means any safe and effective material(s) for use in the compositions of the present invention. Such material(s) include thickening agents, humectants, ionic active ingredients, buffering agents, anticalculus agents, abrasive polishing materials, peroxide sources, alkali metal bicarbonate salts, surfactants, titanium dioxide, coloring agents, flavor systems, sweetening agents, antimicrobial agents, herbal agents, desensitizing agents, stain reducing agents, and mixtures thereof.

In accordance with the preferred embodiments of the present invention, a dentifrice containing both zinc and triclosan can be formulated having different and complementary methods of action, and this can be achieved by use of a dentifrice gum system which includes, e.g., xanthan gum. The dentifrice can employ high levels of a soluble or sparingly soluble zinc active, for example, by using zinc citrate at a relatively high level of from 1 to 2% by weight, based on the weight of the composition. A particularly preferred amount of zinc citrate for use against plaque and gingivitis is 2% by weight, based on the weight of the composition. However, this higher level of soluble zinc may present flavor issues to consumers due to an increase in astringency.

Without being bound by any theory, the present inventors believe that the addition of a relatively low amount of chelating agent to sequester zinc ions may reduce the solubility/astringency of the formulation to such a level that the efficacy is not significantly impacted, but the taste is improved with consumers. Suitable chelating agents may be selected that prove to be triclosan compatible, as evidenced by triclosan stability upon aging and triclosan bioequivalence.

One example of the present invention is a dentifrice composition including an orally acceptable vehicle; an antibacterial agent, e.g., a halogenated diphenyl ether; a soluble zinc salt; and a chelating agent.

A wide variety of antibacterial agents have been suggested in the art to retard plaque formation and the oral infections and dental disease associated with plaque formation. For example, halogenated hydroxydiphenyl ether compounds such as triclosan are well known to the art for their antibacterial activity and have been used in oral compositions to counter plaque formation by bacterial accumulation in the oral cavity.

Halogenated diphenyl ether antibacterial compounds that are useful for the preparation of the compositions of the present invention, based on considerations of antiplaque effectiveness and safety, include 2,4,4′-trichloro-2′-hydroxy-diphenyl ether (triclosan) and 2,2′-dihydroxy-5,5′-dibromo-diphenyl ether. In one embodiment, the antibacterial compound is 2,4,4′-trichloro-2′-hydroxy-diphenyl ether (“Triclosan”).

Non-limiting examples of other suitable antibacterial compounds include phenol and its homologs, mono and polyalkyl and aromatic halophenols, resorcinol and its derivatives and bisphenolic compounds. Such phenolic compounds are fully disclosed in U.S. Pat. No. 5,368,844. Phenolic compounds include n-hexyl resorcinol and 2,2′-methylene bis (4-chloro-6-bromophenol).

The halogenated diphenyl ether or phenolic antibacterial compound is present in the oral composition of the present invention in an effective therapeutic amount. In one embodiment, the effective therapeutic amount ranges of 0.05 wt. % to 2 wt. % based on the weight of the composition. In another embodiment, the effective therapeutic amount ranges of 0.1 wt. % to 1% wt. % based on the weight of the oral composition.

The effectiveness of the antibacterial agent is dependent upon its delivery to and uptake by teeth and soft tissue areas of the gums. The invention therefore can also contain an antibacterial agent and an adherent agent.

An antibacterial enhancing agent may also be included in combination with antibacterial agents such as triclosan. One particularly preferred class of antibacterial enhancing agents for triclosan includes 1:4 to 4:1 copolymers of maleic anhydride or acid with another polymerizable ethylenically unsaturated monomer. For example, one typical maleic anhydride copolymer includes a methyl vinyl ether/maleic anhydride copolymer having a molecular weight (“M. W.”) ranging from 30,000 to abut 5,000,000 g/mole, or from 30,000 to 500,000 g/mole. These copolymers are commercially available, for example, under the trademark Gantrez, including Gantrez AN 139 (M. W. 500,000 g/mole), AN 119 (M. W. 250,000 g/mole); and Gantrez S-97 Pharmaceutical Grade (M. W. 700,000 g/mole), of ISP Corporation. In one aspect, the maleic anhydride copolymer typically comprises a methyl vinyl ether/maleic anhydride copolymer having a molecular weight ranging from 30,000 to abut 1,000,000 g/mole.

The compositions of the present invention further comprise at least one zinc ion source. The zinc ion source can be a soluble or a sparingly soluble compound of zinc. Zinc ions have been found to help in the reduction of gingivitis, plaque, sensitivity, and improved breath benefits.

Zinc ions are derived from the metal ion source(s) found in the dentifrice composition in an effective amount. An effective amount is defined as from at least 1000 ppm zinc ion, preferably 2,000 ppm to 15,000 ppm. More preferably, zinc ions are present in an amount from 3,000 ppm to 13,000 ppm and even more preferably from 4,000 ppm to 10,000 ppm. This is the total amount of zinc ions that is present in the compositions for delivery to the tooth surface. The zinc ion source(s) will be present in an amount of from 0.25% to 11%, by weight of the final composition. Preferably, the zinc ion sources are present in an amount of from 0.4 to 7%, more preferably from 0.45% to 5%.

Examples of suitable zinc ion sources are zinc oxide, zinc sulfate, zinc chloride, zinc citrate, zinc lactate, zinc acetate, zinc gluconate, zinc malate, zinc tartrate, zinc carbonate, zinc phosphate, and other salts listed in U.S. Pat. No. 4,022,880. A zinc salt, e.g., may be present in an amount of from 0.5 to 2.5 wt % based on the weight of the composition, typically from 1 to 2 wt % based on the weight of the composition.

In a further example, the present invention includes a chelating agent, e.g., gluconate, citrate, tartrate, anionic polymeric carboxylate, polyvinyl phosphonate, or phytate. Preferably, the chelating agent is sodium phytate. The chelating agent may be present in an amount of up to 1 wt % based on the weight of the composition.

In one embodiment, the selected chelating agent is dodecasodium phytate, a Generally Regarded as Safe “GRAS” ingredient derived from inositol. Other chelating agents that are compatible with triclosan can also be utilized as described in the invention. Some examples of these chelating agents are gluconates, citrates, and tartrates, rather than, e.g., polyphosphates (which are not triclosan compatible).

Anionic polymeric carboxylates and polyvinyl phosphonates may additionally be helpful in sequestering free zinc. Any chelating agents under consideration should preferably not sequester zinc ions to the extent that the product is no longer efficacious, nor should they be strong enough (or plentiful enough in formulation) to sequester calcium and promote demineralization of enamel.

In yet another example, the present invention includes a polysaccharide thickening agent, e.g., xanthan gum and hydroxyethyl cellulose. Thickening agents provide the dentifrice with the required rheological properties, so that the dentifrice can be stored in a dispensing container over a period of time and thereafter reliably dispensed therefrom by the user. The dentifrice must have the correct viscosity not only to be dispensed but also to exhibit an acceptable consistency within the mouth during tooth brushing. Typical thickening agents include modified celluloses, such as carboxymethyl cellulose (CMC), and other polysaccharide or gum components. Preferably, the polysaccharide thickening agent consists of xanthan gum which is present in an amount of from 0.1 to 1.5 wt % based on the weight of the composition, preferably from 0.5 to 1 wt % of the composition. However, minor amounts of additional thickeners may be present, for example carrageenan, gum tragacanth, starch, polyvinylpyrollidione, hydroxyethypropyl cellulose, hydroxybutyl methyl cellulose, hydroxypropyl methylcellulose, hydroxyethyl cellulose, sodium carboxymethyl cellulose (sodium CMC) and colloidal silica. In one embodiment, the thickener concentration ranges of 0.1 wt. % to 5 wt. % based on the weight of the composition. In another embodiment, the thickener concentration ranges of 0.5 wt. % to 2 wt. % based on the weight of the composition.

In a further example, the invention includes a dentifrice composition comprising an orally acceptable vehicle; triclosan; a soluble zinc salt; and a chelating agent consisting of sodium phytate. The chelating agent is present in an amount of from 0.1 to 0.5 wt % based on the weight of the composition, the soluble zinc salt is present in an amount of from 0.5 to 2.5 wt % based on the weight of the composition, and the triclosan is present in an amount of from 0.1 to 1 wt % based on the weight of the composition.

In another example, the present invention includes a method for the treatment and prevention of bacterial plaque accumulation comprising: administering to the oral cavity a dentifrice composition according to the invention.

The present compositions comprise essential components, as well as optional components. The essential and optional components of the compositions of the present invention are described in the following paragraphs.

In preparing the present compositions, it is desirable to add one or more aqueous carriers to the compositions. Such materials are well known in the art and are readily chosen by one skilled in the art based on the physical and aesthetic properties desired for the compositions being prepared. Aqueous carriers typically comprise from 40% to 99%, preferably from 70% to 98%, and more preferably from 90% to 95%, by weight of the dentifrice composition.

In the preparation of an oral composition in accordance with the practice of the present invention, an orally acceptable vehicle including a water-phase with humectant is present. The humectant includes one or more of glycerin, sorbitol, propylene glycol and mixtures thereof. In one embodiment, water is present in amount of at least 10 wt. % based on the weight of the composition. In another embodiment, water is present in an amount of at least 30 wt. % to 60 wt. % based on the weight of the composition. In yet another embodiment, the humectant concentration typically totals 40-60 wt. % of the oral composition.

Dentifrice compositions such as toothpastes and gels also typically contain polishing materials. In one embodiment, the polishing material includes crystalline silica, having a particle size of up to 20 microns, such as commercially available Zeodent 115, or Zeodent 165, silica gel or colloidal silica. In another embodiment, the polishing material includes compositions such as complex amorphous alkali metal aluminosilicates, hydrated alumina, sodium metaphosphate, sodium bicarbonate, calcium carbonate, calcium pyrophosphate, dicalcium phosphate and dicalcium phosphate dihydrate. In one embodiment, the polishing material is included in semi-solid or pasty dentifrice compositions, of the present invention, in an amount of 15 wt. % to 60 wt. %. In another embodiment, the composition of the present invention includes polishing material having concentrations ranging of 20 wt. % to 55 wt. % based on the weight of the composition.

The oral composition may also contain a source of fluoride ions, or fluoride-providing compound, as an anti-caries agent. In one embodiment, the fluoride ion composition is provided in an amount sufficient to supply fluoride ions ranging from 25 ppm to 5,000 ppm of the oral composition In another embodiment, the fluoride ion composition is provided in an amount sufficient to supply fluoride ions ranging from 500 to 1500 ppm of the oral composition. Representative fluoride ion providing compounds include inorganic fluoride salts, such as soluble alkali metal salts, for example, sodium fluoride, potassium fluoride, sodium fluorosilicate, ammonium flourosilicate and sodium monofluorphosphate, as well as tin fluorides, such as stannous fluoride and stannous chloride.

Any suitable flavoring or sweetening material may also be employed in the preparation of the oral compositions of the present invention. Examples of suitable flavoring constituents include flavoring oils, e.g. oil of spearmint, peppermint, wintergreen, clove, sage, eucalyptus, marjoram, cinnamon, lemon, orange, and methyl salicylate. Suitable sweetening agents include sucrose, lactose, maltose, xylitol, sodium cyclamate, aspartyl phenyl alanine methyl ester, saccharine and the like. Suitably, flavor and sweetening agents may each or together constitute 0.1 wt. % to 5 wt. % of the oral composition.

Various other materials may be incorporated in the oral preparations of this invention such as whitening agents, including urea peroxide, calcium peroxide, and hydrogen peroxide, preservatives, vitamins such as vitamin B6, B 12, E and K, silicones, chlorophyll compounds and potassium salts for the treatment of dental hypersensitivity such as potassium nitrate and potassium citrate. These agents, when present, are incorporated in the compositions of the present invention in amounts which do not substantially adversely affect the properties and characteristics desired.

The dispenser for the dentifrice compositions may be a tube, pump, or any other container suitable for dispensing toothpaste.

In practicing the present invention, the user need only apply the dentifrice composition herein, to the tooth surfaces of a human or lower animal, in the areas desired, in order to obtain a desired effect, e.g., whitening, breath freshening, caries prevention, pain relief, gum health, tartar control, etc. The compositions may also be applied to other oral cavity surfaces, such as the gingival or mucosal tissues, although it is believed that the benefits are best achieved when the dentifrice compositions are applied to the teeth. The dentifrice composition may contact the tooth and/or oral cavity surface either directly, or indirectly; however, it is preferred that the dentifrice composition be directly applied. The dentifrice composition may be applied by any means, but is preferably applied with a brush or by rinsing with a dentifrice slurry.

The manufacture of the oral composition of the present invention is accomplished by any of the various standard techniques for producing such compositions. To make a dentifrice, a vehicle is prepared containing humectant, for example, one or more of glycerin, glycerol, sorbitol, and propylene glycol, thickener agents and antibacterial agent such as triclosan, and the vehicle and any surfactants are added, followed by blending in of a polishing agent, as well as fluoride salts, with the pre-mix. Finally, flavoring agent is admixed and the pH is adjusted to between 6.8 and 7.

The following examples are further illustrative of the present invention, but it is understood that the invention is not limited thereto. All amounts and proportions referred to herein and in the appended claims are by weight, unless otherwise indicated.

### EXPERIMENTAL EXAMPLES

### Example 1:

Flavor assessment of prototype batches indicate that the preferable level of sodium phytate for use in the dentifrice formulation of the present invention is 0.5% or less. The following table indicates the level (and long term stability) of soluble zinc in three prototype formulas (0.25 and 0.5% sodium phytate):

TABLE 1
Zinc Solubility/Stability in Presence of Phytate

| | | % Soluble | Soluble Zinc @ | | |
|---|---|---|---|---|---|
| | % Sodium | zinc initial | 40 C./75% RH | | |
| | Phytate | value | 1 month | 2 month | 3 month |
| Control | 0 | 0.46 | 0.43 | 0.43 | 0.4 |
| Sample 1 | 0.25 | 0.41 | 0.37 | 0.36 | 0.3 |
| Sample 2 | 0.5 | 0.35 | 0.32 | 0.3 | 0.22 |
| Comparative | 0 | 0.29 | 0.2 | 0.14 | 0.15 |
| Sample 3 [2% | | | | | |
| zinc citrate; | | | | | |
| no triclosan] | | | | | |

As shown above, by controlling the solubility of zinc (through chelation of excess zinc ions) the astringency of the formulations should be reduced, thereby making the formulations more consumer appealing and flavor optimization less difficult. At the same time, as shown above, the addition of the phytate does not reduce the soluble zinc below that which has been determined to be efficacious (as compared to Comparative Sample 3, a clinically tested 2% zinc citrate formulation).

The Electron Spectroscopy for Chemical Analysis (ESCA) results for hydroxyapatite (HAP) disks treated with the SnF₂, Zn citrate and phytic acid (inositol hexaphosphate) formulas are shown below. The table below represents the average compositional data for all samples of each treatment. Each sample is analyzed in two separate locations to confirm uniformity of composition. Triplicate samples are analyzed for each treatment, with the exception of the untreated control.

| | | | Base | Base | Base |
|---|---|---|---|---|---|
| | | | Formula | Formula | Formula |
| | | Base | Zinc w/ | Zinc w/ | Zinc w/ |
| | Base | formula | 0.25% | 0.5% | 1.0% |
| Formula | Formula | with Zinc | Phytic | Phytic | Phytic |
| Water | 6 | 6 | 6 | 6 | 6 |
| Saccharin | 0.3 | 0.3 | 0.3 | 0.3 | 0.3 |
| Stannous Fluoride | 0.454 | 0.454 | 0.454 | 0.454 | 0.454 |
| Citric Acid | 0.6 | 0.6 | 0.6 | 0.6 | 0.6 |
| Trisodium Citrate | 3 | 3 | 3 | 3 | 3 |
| Zinc Citrate | 0 | 2 | 2 | 2 | 2 |
| Glycerin | 35.857 | 33.857 | 33.607 | 33.857 | 32.857 |
| Propylene glycol | 0.5 | 0.5 | 0.5 | 0.5 | 0.5 |
| Sodium CMC | 1.1 | 1.1 | 1.1 | 1.1 | 1.1 |
| Carrageenan | 0.5 | 0.5 | 0.5 | 0.5 | 0.5 |
| Titanium Dioxide | 0.75 | 0.75 | 0.75 | 0.75 | 0.75 |
| Sorbitol | 15.539 | 15.539 | 15.539 | 15.039 | 15.539 |
| Gantrez S97 | 15 | 15 | 15 | 15 | 15 |
| polymer | | | | | |
| Sodium | 1.2 | 1.2 | 1.2 | 1.2 | 1.2 |
| Hydroxide | | | | | |
| Silica | 16.5 | 16.5 | 16.5 | 16.5 | 16.5 |
| Flavor | 1.2 | 1.2 | 1.2 | 1.2 | 1.2 |
| Sodium lauryl | 1.5 | 1.5 | 1.5 | 1.5 | 1.5 |
| sulfate | | | | | |
| Phytic Acid | 0 | 0 | 0.25 | 0.5 | 1 |
| | 100 | 100 | 100 | 100 | 100 |

ESCA Surface Composition - Average of All Disks Studied

| | Atomic Percent | | | | | | | | | | Ratio |
|---|---|---|---|---|---|---|---|---|---|---|---|
| Sample | C | O | N | Ca | P | Mg | Na | F | Zn | Sn | P/Ca |
| HAP Control | 15.24 | 53.33 | 0.00 | 15.54 | 11.19 | 4.36 | 0.35 | — | — | — | 0.72 |
| Saliva Control | 34.38 | 39.15 | 8.08 | 9.42 | 7.62 | 1.36 | 0.00 | — | — | — | 0.81 |
| 2:1 Phytic acid | 27.24 | 42.35 | 5.08 | 9.29 | 8.89 | 0.52 | 6.69 | — | — | — | 0.96 |
| Base w/ SnF2 | 25.67 | 47.70 | 1.09 | 11.41 | 8.82 | 1.88 | 2.70 | 0.44 | — | 0.30 | 0.77 |
| Base w/ SnF2/2% Zn | 29.60 | 46.86 | 2.58 | 9.28 | 7.27 | 1.17 | 2.01 | 0.30 | 0.67 | 0.27 | 0.78 |
| citrate | | | | | | | | | | | |
| Base w/ SnF2/2% Zn | 30.69 | 45.58 | 1.82 | 9.47 | 7.68 | 1.19 | 2.12 | 0.37 | 0.79 | 0.31 | 0.81 |
| citrate/0.25% Phytic | | | | | | | | | | | |
| Acid | | | | | | | | | | | |
| Base w/ SnF2/2% Zn | 31.76 | 44.80 | 1.43 | 9.04 | 7.75 | 1.54 | 2.42 | 0.30 | 0.76 | 0.22 | 0.86 |
| citrate/0.5% Phytic | | | | | | | | | | | |
| Acid | | | | | | | | | | | |
| Base w/ SnF2/2% Zn | 32.84 | 43.61 | 1.99 | 9.14 | 7.66 | 1.19 | 2.32 | 0.30 | 0.74 | 0.20 | 0.84 |
| citrate/1.0% Phytic | | | | | | | | | | | |
| Acid | | | | | | | | | | | |

The composition of the control HAP disks was typical for untreated HAP surfaces. The C concentration was low, with N absent from the surface. Ca, P and Mg were all observed in significant quantities and the P/Ca was consistent with that for HAP disks. The saliva treated HAP exhibited an increase in C and significant surface N, indicating the presence of surface proteins on the disk. The Ca and P levels were reduced due to the presence of the coating. The P/Ca ratio also increased slightly due to phosphate present in the saliva.

The disk treated with phytic acid also exhibited increases in C and N due to the presence of saliva proteins on the surface. Ca and P were reduced due to the protein coating. In addition, a significant amount of Na was observed on the disk. The Na originates from the phytic acid, since the acid raw material has been completely neutralized and is actually the Na salt of phytic acid. The P ESCA peak for the phytic acid was not shifted from the P peak of the HAP, so direct detection of phytic acid on HAP is not possible by ESCA. A significant increase in P/Ca was observed, however, reflecting the deposition of the phytic acid on the surface. Thus detection of phytic acid on HAP by ESCA is only indirectly possible through an increase in P/Ca.

The samples treated with the various toothpaste formulas exhibited increases in C and N, relative to the untreated disks, from organics in the paste and the saliva. The N levels for the disks were less than that for the saliva control, indicating much less saliva protein is present on the treated disks. The Ca and P levels for the treated disks were lower than those for the untreated disks, due to surface coverage by the organics. The Ca and P concentrations for the base/SnF₂ treated disks were higher than those for the other treated disks. The C concentration was also lower for the base/SnF₂ disks than for the other treated disks. This difference between the base/SnF₂ disks and the other disks may be due to the presence of citrate and/or phytic acid on the surfaces of the disks that contain these components. F was detected on the surfaces of all the treated samples. The F concentration was highest for the HAP treated with the base/SnF₂ formula. The F concentrations for the other disks were similar, within the variation of the data. The data did not suggest that the phytic acid had an influence on F deposition on the disks. Zn was detected on the surfaces of the disks treated with the Zn citrate containing formulas. The concentrations of Zn on the disks varied somewhat from sample to sample, thus indicating that each Zn containing formula deposits similar amounts of Zn on the disk surfaces. The phytic acid appeared to have no influence on Zn deposition. Sn was detected on all the treated samples as well. The Sn concentrations were similar for the HAP treated with formulas that did not contain phytic acid and the formula that contained 0.25% phytic acid. The Sn level decreased slightly for the HAP treated with the 0.5% or 1% phytic acid formulas. The data also suggest that Sn deposition decreases with increasing phytic acid concentration. Thus phytic acid does have an effect on Sn deposition for formulas containing greater than 0.25% phytic acid. Finally, the P/Ca ratios for the disks treated with the phytic acid formulas were slightly higher than those for the disks treated with formulas that did not contain phytic acid. This result coupled with the slightly higher C concentrations for the HAP treated with phytic acid formulas may suggest deposition of phytic acid on the disk surfaces.

Static Secondary Ion Mass Spectroscopy (sSIMS) is used to characterize the reference and treated HAP disks to provide additional evidence for deposition of phytic acid on the surfaces. The molecular weight for phytic acid exceeds the mass range for the SIMS instrument, however, so deposition of phytic acid cannot be determined by detection of the molecular ion. Instead, phytic acid detection needs to be accomplished through observation of peaks in the mass spectra from fragments of the phytic acid molecule. Study of the reference and treated disks revealed a dramatic increase in the negative ion phosphate peaks PO³⁻ and PO⁴⁻ for HAP treated with the phytic acid formulas compared to the disks treated with formulas that did not contain phytic acid. Mass peaks more specific to fragments of the phytic acid molecule were not observed. Thus the increase in phosphate ion peak intensity for samples treated with the phytic acid formulas relative to the phytic acid free reference samples is used to provide additional evidence for phytic acid deposition. Since formulas with phytic acid concentrations ranging from 0 to 1% were used in the study, measurement of phosphate peak intensity with increasing phytic acid concentration in the formula was also conducted. A strong SO³⁻ peak was also observed in the negative ion mass spectra from residual surfactant on the disk surfaces. This sulfate peak was consistent in intensity for each sample, and was thus used as a reference peak for phosphate intensity measurements. A table of the average PO³⁻/SO³⁻ peak intensity ratios for the treated disks versus phytic acid concentration in the formulas is shown below. The data shows an increase in intensity ratio with increasing phytic acid concentration. Thus the table shows that phytic acid is depositing on the surface and the amount of deposition increases with increasing concentration in the formula.

sSIMS of HAP Disks

| Phytic Acid Concentration | |
|---|---|
| Weight % | PO³⁻/SO³⁻ |
| 0 | 0.18 |
| 0.25 | 0.42 |
| 0.5 | 0.5 |
| 1 | 0.69 |

The ESCA results indicate deposition of Sn and Zn on the disk surfaces. Phytic acid in the formula did not influence Zn deposition, however, Sn deposition decreased with increasing phytic acid content in the paste. Both ESCA and sSIMS suggest that phytic acid also deposited on the disks, with the sSIMS data indicating that deposition increases with increasing phytic acid content in the paste.

What is claimed is:

1. A dentifrice composition comprising:
a. an orally acceptable vehicle;
b. a halogenated diphenyl ether;
c. a soluble zinc salt; and
d. a chelating agent, wherein the chelating agent is at least one chelating agent chosen from polyvinyl phosphonates, and phytates, wherein the chelating agent is present in an amount up to 1% based on the weight of the composition.

2-18. (canceled)

19. The composition of claim 1, wherein the chelating agent comprises dodecasodium phytate or sodium phytate.

20. The composition of claim 19, wherein the soluble zinc salt is present in an amount of 0.5 to 2.5 wt % based on the weight of the composition.

21. The composition of 20, wherein the soluble zinc salt comprises at least one zinc salt chosen from zinc citrate, zinc acetate, zinc lactate, zinc chloride, and zinc gluconate.

22. The composition of 21, wherein the soluble zinc salt comprises zinc citrate.

23. The composition of claim 21, wherein
the chelating agent is present in an amount of 0.1 to 1 wt % based on the weight of the composition.

24. The composition of claim 23, wherein the halogenated diphenyl ether is triclosan.

25. The composition of claim 24, wherein the dentifrice composition further comprises stannous fluoride and a methyl vinyl ether/maleic anhydride copolymer having a molecular weight ranging from 30,000 to 1,000,000 g/mole.

26. A method for the treatment and prevention of bacterial plaque accumulation comprising: administering to the oral cavity a dentifrice composition according to claim 1.
